(19) United States
(12) Patent Application Publication
(10) Pub. No.: US 2018/0235210 A1
(43) Pub. Date: Aug. 23, 2018

(54) ANTISENSE COMPOSITIONS TARGETING ERWINIA SPECIES AND METHODS OF USING THE SAME

(71) Applicants: T3 Bioscience, LLC, Mequon, WI (US); The Connecticut Agricultural Experiment Station, New Haven, CT (US)

(72) Inventors: Quan Zeng, Cheshire, CT (US); Ching-Hong Yang, Mequon, WI (US)

(73) Assignees: T3 Bioscience, LLC, Mequon, WI (US); The Connecticut Agricultural Experiment Station, New Haven, CT (US)

(21) Appl. No.: 15/902,040

(22) Filed: Feb. 22, 2018

Related U.S. Application Data
(60) Provisional application No. 62/462,086, filed on Feb. 22, 2017.

(51) Int. Cl. A01N 25/10; A01N 63/00; A01N 57/12; C12N 15/113; C07K 19/00; C07K 7/06; C07K 7/08; C07K 14/00

(52) U.S. Cl. CPC A01N 25/10; A01N 63/00; A01N 57/12; C12N 15/1137; C12N 15/113; C12N 2310/3181; C07K 7/06; C07K 7/08; C07K 14/00; C12N 2310/11; C12N 2310/3513; C07K 19/00

(57) ABSTRACT

The invention generally relates to antisense compositions targeting Erwinia species and methods of using the same. More specifically, the invention relates in part to compositions including a cell penetrating peptide (CPP) linked to an antisense polynucleotide complementary to a target sequence in an RNA expressed from an essential gene in an Erwinia species and methods of using such compositions to inhibit the growth of an Erwinia species on a plant.

### CROSS-REFERENCE TO RELATED PATENT APPLICATIONS

The present application claims the benefit of priority to United States Provisional Patent Application No. 62/462,086, filed on Feb. 22, 2017, the content of which is incorporated herein by reference in its entirety.

### STATEMENT REGARDING FEDERALLY SPONSORED RESEARCH

This invention was made with United States government support awarded by the United States Department of Agriculture (“USDA”) grant numbers 2016-67030-24856 and CONH00650. The United States has certain rights in this invention.

### SEQUENCE LISTING

This application is being filed electronically via EFS-Web and includes an electronically submitted Sequence Listing in .txt format. The .txt file contains a sequence listing entitled “2018-02-21_6422-00008_ST25.txt” created on Feb. 21, 2018 and is 5,771 bytes in size. The Sequence Listing contained in this .txt file is part of the specification and is hereby incorporated by reference herein in its entirety.

### INTRODUCTION

Fire blight, caused by the bacterial pathogen Erwinia amylovora, is one of the most serious diseases of apple and pear in the United States and worldwide. Fire blight infection can occur in flowers, leaves, shoots, and fruits, resulting in yield reduction; the pathogen also can spread systemically through trees to the rootstock, ultimately resulting in tree death. Annual losses to fire blight and costs of control in the United States are estimated at over $100 million.

The management of fire blight is challenging due to the availability of limited control options. As the pathogens enter plants through the natural opening of the flowers, antibiotic spray applications during bloom are the most effective and widely used control method for fire blight. Streptomycin is the most effective antibiotic targeting E. amylovora, and has been used for fire blight management since the 1950s. The intensive, long-term use of streptomycin, however, has resulted in the development of streptomycin resistance in the E. amylovora population. Since its original report in California in 1972, streptomycin resistance has been observed in most major apple-producing regions in the United States. In addition, agricultural application of streptomycin also raises significant concerns for the potential selection of antibiotic resistant bacteria in the environment, and the potential impact to human health. Besides antibiotics, copper bactericides and other biological control products are also used for fire blight management, however the use of these materials is limited by their inconsistent control efficacy and copper use can also result in phytotoxicity. Because of these reasons, developing effective control alternatives for fire blight has become an urgent need for sustainable apple and pear production in the United States.

### SUMMARY

In one aspect of the present invention, antimicrobial compositions are provided. The antimicrobial compositions may include a cell penetrating peptide (CPP) linked to an antisense polynucleotide complementary to a target sequence in an RNA expressed from an essential gene in an Erwinia species.

In a further aspect, agricultural compositions are provided. The agricultural compositions may include any one of the antimicrobial compositions described herein and a carrier, a biocontrol agent, an additional active ingredient or any combination thereof.

In a still further aspect, methods for inhibiting the growth of an Erwinia species on a plant are provided. The methods may include contacting the plant with an effective amount of any one of the compositions described herein to inhibit the growth of the Erwinia species on the plant.

### BRIEF DESCRIPTION OF DRAWINGS

FIGS. 1A-1C show the effect of PNA-CPP on the growth of E. amylovora Ea110 in LB broth. FIG. 1A shows E. amylovora treated with anti-acpP PNA. FIG. 1B shows E. amylovora treated with CPP conjugated PNA: anti-acpP-CPP1, anti-acpP-CPP2 and anti-acpP-CPP3. FIG. 1C shows E. amylovora treated with CPP1 conjugated PNA containing random sequence (non-target-CPP1).

FIG. 2 shows the growth inhibition of E. amylovora Ea110 caused by a range of different concentrations of anti-acpP-CPP1 and streptomycin in LB broth.

FIG. 3 shows Northern blot detection of the expression levels of acpP in E. amylovora Ea110 treated with anti-acpP-CPP1 and streptomycin.

FIG. 4 shows the effect of anti-acpP-CPP1 on the viability of E. amylovora Ea110. FIG. 4A shows the number of viable E. amylovora cells recovered on LB plates from the anti-acpP-CPP1 and streptomycin treatment. FIG. 4B shows E. amylovora stained with fluorescent dye SYTO 9 and propidium after 6 hours of treatment with anti-acpP-CPP1 and water. Heat-treated cells (80° C. for 10 mins) were included as a control.

FIG. 5 shows the amount of E. amylovora Ea110 cells on detached apple stigmas after treatment with anti-acpP-CPP1 and streptomycin.

FIG. 6 shows an alignment of the acpP start codon sequences of E. amylovora with other plant associated bacteria (SEQ ID NOS: 13-18). Sequences were obtained from NCBI database based on the availability of the bacterial genomes. The −5 to +5 sequences where the PNA-CPP targets are underlined, with mismatches heighted in red and start codon in bold. The open reading frame of acpP was shaded in light blue. The binding of PNA-CPP to acpP in E. amylovora (SEQ ID NO: 1 and 2) was also illustrated. All sequences were written in a 5′ to 3′ order unless labeled.

FIG. 7 shows the growth of E. amylovora upon the addition of CPP-PNA targeting different essential genes.

### DETAILED DESCRIPTION

Here, in the non-limiting Examples, the present inventors have developed antisense antimicrobial compositions against Erwinia species such as Erwinia amylovora and have used these compounds to inhibit E. amylovora growth on plants. They determined that a 10-nucleotide oligomer of peptide nucleic acid (PNA) targeting an essential gene, such as acpP or fabA, is able to cause complete growth inhibition of E. amylovora. They found that conjugation of a cell penetrating peptide (CPP) to the PNA is essential for the antimicrobial effect, with CPP1 (SEQ ID NO: 2) being the most effective against E. amylovora. The minimal inhibitory concentration (MIC) of an anti-acpP-CPP1 (2.5 μM) (SEQ ID NO: 1 linked to SEQ ID NO: 2) is comparable to the MIC of streptomycin (2 μM). Examination of the antimicrobial mechanisms demonstrated that anti-acpP-CPP1 caused dose-dependent reduction of acpP mRNA in E. amylovora upon treatment and resulted in cell death (bactericidal effect). Anti-acpP-CPP1 (100 μM) was also able to effectively limit the pathogen growth on stigmas of apple flowers, although less effective than streptomycin. Finally, unlike streptomycin that does not display any specificity in inhibiting pathogen growth, the antimicrobial effect of anti-acpP-CPP1 is restricted to E. amylovora and a few other bacterial species, but does not affect the growth of most non-pathogenic, flower-associated microorganisms. In summary, the present inventors demonstrate that PNA-CPP can cause an effective, specific antimicrobial effect against Erwinia species such as E. amylovora and may provide the basis for a novel approach for controlling Erwinia caused plant diseases such as fire blight.

In one aspect of the present invention, antimicrobial compositions are provided. The antimicrobial compositions may include a cell penetrating peptide (CPP) linked to an antisense polynucleotide complementary to a target sequence in an RNA expressed from an essential gene in an Erwinia species. The essential gene may be acpP or fabA and the antisense oligonucleotide may be directed to the a portion of the gene near the start codon.

The terms “polynucleotide,” “nucleotide sequence,” “polynucleotide sequence,” “nucleic acid” and “nucleic acid sequence” refer to a nucleotide, oligonucleotide, polynucleotide (which terms may be used interchangeably), or any fragment thereof. These phrases may refer to, without limitation, DNA, RNA, peptide nucleic acids (PNAs), phosphorodimidate mopholino oligomers (PMOs), locked nucleic acids (LNA) or phosphorothioate oligonucleotides (PS-ODNs) of natural, or synthetic origin.

The antisense polynucleotide may be any polynucleotide sequence that is complementary to a target sequence in an RNA expressed from an essential gene in an Erwinia species. The antisense polynucleotide may be an unmodified polynucleotide or may be a modified polynucleotide that, for example, contains modifications that increase the stability of the polynucleotide. Polynucleotide modifications to, for example, protect the polynucleotide from nuclease degradation and/or increase the stability of the polynucleotide in an environment (i.e., cellular or otherwise) are well-known in the art.

In some embodiments, the antisense polynucleotide may be a peptide nucleic acid (PNA), a phosphorodimidate mopholino oligomer (PMO), phosphorothioate oligonucleotides (PS-ODN) or antisense 2′-deoxy, 2′-fluoroarabino nucleic acid (2′ F-ANA) oligonucleotides. In PNAs, the sugar-phosphate backbone of DNA/RNA is replaced with a pseudopeptide backbone, while nearly identical geometry and spacing of the bases is retained. See, e.g., Bal and Luo, 2012. This modification not only significantly enhances the stability, but also increases PNA-RNA binding affinity, as RNA is negatively charged but PNA is electrically neutral. PMOs are polynucleotides having a backbone of methylenemorpholine rings and phosphorodiamidate linkages. PS-ODNs are polynucleotides having phosphorothioate bonds (rather than phosphodiester bonds) in the sugar-phosphate backbone. 2′ F-ANA uses 2′-deoxy, 2′-fluoro-β-d-arabinonucleic acid to replace deoxyribose in the oligo chain. Suitably, the antisense polynucleotide includes a peptide nucleic acid (PNA).

Additional common nucleotide base modifications may be used to modifiy the antisense polynucleotide in accordance with the present invention. Such modifications include, without limitation, 2′-O-Methyl bases, 2′-Fluoro bases, 2′ Amino bases, inverted deoxythymidine bases, 5′ modifications, and 3′ modifications. These modifications may make the oligonucleotide more stable or less susceptible to degradation.

The antisense polynucleotide may include 6, 7, 8, 9, 10, 11, 12, 13, 14, 15, 16, 17, 18, 19 20, 25, 30, 35, 40, 45, 50, 55 or more nucleotides. In some embodiments, the antisense polynucleotide includes or consists of 6 to 20 nucleotides.

The antisense polynucleotide may be complementary to 75%, 80%, 85%, 90%, 95%, 97%, 98%, 99%, or 100% (% sequence complementarity) of a target sequence in an RNA expressed from an essential gene in an Erwinia species. For example, if the antisense polynucleotide includes 10 nucleotide bases, all 10 nucleotide bases may be complementary to the target sequence (100% sequence complementarity). The antisense polynucleotide may also include 1, 2, 3, or 4 nucleotide bases that do not base pair with the target sequence. Preferably, the antisense polynucleotide is complementary to 100% of the target sequence.

In some embodiments, the antisense polynucleotide may include SEQ ID NO: 1 (anti-acpP PNA sequence) or a fragment consisting of at least 6, 7, 8, or 9 nucleotides thereof. In some embodiments, the antisense polynucleotide may include SEQ ID NO: 19 (anti-fabA PNA sequence) or a fragment consisting of at least 6, 7, 8, or 9 nucleotides thereof.

The essential gene may be any gene in an Erwinia species which, when expressed, is required for viability. The essential gene may encode an mRNA or another type of RNA such as a ribosomal RNA. Suitable essential genes include, without limitation, acpP, fabA, inhA, ompA, 16s RNA gene, or adk.

The target sequence in the RNA expressed from the essential gene may be any sequence in the RNA expressed from the essential gene including at least 6 consecutive nucleotides. Suitably, in messenger RNAs expressed from essential genes the target sequence includes the start codon, the Shine-Dalgarno (SD) sequence, or both.

The cell penetrating peptide (CPP) may be any polypeptide having less than 50, 40, 30, or 20 amino acids that can penetrate cell membranes and deliver conjugated cargoes into cells. See, e.g., Abes et al., 2007; Bal and Luo, 2012. Conjugation of CPPs with PNAs, for example, has been demonstrated to significantly increase PNA entry into bacterial cells. See, e.g., Rasmussen et al., 2007; Zatsepin et al., 2005. Two features of CPPs that are required for cell penetrating ability include amphipathicity and positive charge. These characteristics are acquired by synthesizing CPPs with an alternation of cationic amino acid residues and nonpolar residues. Suitably, the CPP includes SEQ ID NO: 2 (CPP1), SEQ ID NO: 3 (CPP2), SEQ ID NO: 4 (CPP3), or a combination thereof.

Fragments and/or variants of SEQ ID NO: 2 (CPP1), SEQ ID NO: 3 (CPP2), SEQ ID NO: 4 (CPP3), or combinations thereof may also be used in accordance with the present invention. As used herein, a “fragment” is a portion of an amino acid sequence which is identical in sequence to but shorter in length than SEQ ID NO: 2 (CPP1), SEQ ID NO: 3 (CPP2), or SEQ ID NO: 4 (CPP3) (“reference CPP sequences”). A fragment may comprise up to the entire length of the reference CPP sequence, minus at least one amino acid residue. For example, a fragment may comprise from 5 to 10 contiguous amino acid residues of a reference CPP sequence, respectively. In some embodiments, a fragment may comprise at least 5, 6, 7, 8, 9, 10, 15, or 20, contiguous amino acid residues of a reference CPP sequence. A fragment may include an N-terminal truncation, a C-terminal truncation, or both truncations relative to the full-length reference CPP sequence. Preferably, a fragment of a reference protein includes amino acid residues required for the cell penetrating activity.

Variants of SEQ ID NO: 2 (CPP1), SEQ ID NO: 3 (CPP2), or SEQ ID NO: 4 (CPP3) may include polypetides having at least 80%, 90%, 95%, 98%, or 99% sequence identity to SEQ ID NO: 2 (CPP1), SEQ ID NO: 3 (CPP2), or SEQ ID NO: 4 (CPP3) (“reference CPP sequences”). The phrases “% sequence identity,” “percent identity,” and “% identity” refer to the percentage of residue matches between at least two amino acid sequences aligned using a standardized algorithm. The amino acid sequences of the reference CPP sequence variants as contemplated herein may include conservative amino acid substitutions relative to the reference CPP sequences. For example, a variant CPP sequence may include conservative amino acid substitutions relative to a reference molecule. “Conservative amino acid substitutions” are those substitutions that are a substitution of an amino acid for a different amino acid where the substitution is predicted to interfere least with the properties of the reference CPP sequence. In other words, conservative amino acid substitutions substantially conserve the structure and the function of the reference polypeptide. Conservative amino acid substitutions generally maintain the amphipathicity and overall positive charge of the CPP sequence. For example, a lysine residue (K) in SEQ ID NO: 2 (CPP1) may be replaced with another positively charged amino acid such as an arginine (R) residue. Preferably, the CPP includes SEQ ID NO: 2 (CPP1).

The cell penetrating peptide (CPP) may be linked to the antisense polynucleotide covalently, noncovalently, and/or via a linker. Suitable linker or spacer moieties include peptides, amino acids, nucleic acids, as well as homofunctional linkers or heterofunctional linkers. Particularly useful linkers for facilating the formation of a covalent bond between an antisense polynucleotide and a CPP are known in the art and may include, without limitation, ethylene glycol linkers (egl), AEEA linkers, or O linkers. Additional linkers that can facilitate formation of a covalent bond between an antisense polynucleotide and a CPP may include N-hydroxysuccinimide (NHS) esters and/or a maleimide.

In some embodiments, the antisense polynucleotide and the CPP are linked noncovalently by a tag system. A tag system includes any group of agents capable of binding one another with a high affinity. Several tag systems are well-known in the art and include, without limitation, biotin/avidin, biotin/streptavidin, or digoxigenin (DIG) systems. In some embodiments, the tag system comprises biotin/avidin or biotin/streptavidin. In such embodiments, the antisense polynucleotide may be modified at either the 5′ or 3′ end to include biotin while the CPP may be modified at the N-terminus or C-terminus to include streptavidin or avidin. Alternatively, the antisense polynucleotide may be modified at either the 5′ or 3′ end to include streptavidin or avidin while the CPP may be modified at the N-terminus or C-terminus to include biotin.

As used herein, the Erwinia species may be any Erwinia species in the Erwinia genus of Enterobacteriaceae bacteria. Suitably, the Erwinia species is a pathogenic Erwinia species and may include, without limitation, Erwinia amylovora and Erwinia pyrifoliae. Erwinia pyrifoliae is within the same genus as Erwinia amylovora and like Erwinia amylovora is a plant pathogen causing, for example, necrotic disease in Asian pears.

In some embodiments, the antimicrobial compositions of the present invention may include SEQ ID NO: 1 linked to SEQ ID NO: 2 (anti-acpP-CPP1), SEQ ID NO: 1 linked to SEQ ID NO: 3 (anti-acpP-CPP2), or SEQ ID NO: 1 linked to SEQ ID NO: 4 (anti-acpP-CPP3). Suitably, the antimicrobial composition includes SEQ ID NO: 1 linked to SEQ ID NO: 2 via an ethylene glycol linker (anti-acpP-CPP1). In some embodiments, the antimicrobial compositions of the present invention may include SEQ ID NO: 19 linked to SEQ ID NO: 2 (anti-fabA-CPP1), SEQ ID NO: 19 linked to SEQ ID NO: 3 (anti-fabA-CPP2), or SEQ ID NO: 19 linked to SEQ ID NO: 4 (anti-fabA-CPP3). Suitably, the antimicrobial composition includes SEQ ID NO: 19 linked to SEQ ID NO: 2 via an ethylene glycol linker (anti-fabA-CPP1).

In another aspect of the present invention, agricultural compositions are provided. The agricultural compositions may include any one of the antimicrobial compositions described herein and a carrier, a biocontrol agent, an additional active ingredient or any combination thereof. An “agricultural composition” is a composition formulated for application to a plant or plant part. An agricultural composition is commonly in liquid form for application by spraying or soaking, but may be in a solid or powder form for rehydration or application by dusting or dry coating. The agricultural composition may be concentrated for dilution in water or other solvent.

The agricultural composition may include a carrier. Carriers may be solid or liquid and may include substances ordinarily employed in formulations applied to plants. Carriers may include nonionic surfactants or ionic sufactants such as cationic or anionic surfactants. Suitable carriers may include, without limitation, regulaid (Kalo), TERMUL® emulsifiers (i.e., TERMUL® 2507, Huntsman), TERWET® adjuvants (i.e., TERWET® 1108 and TERWET® 1109, Huntsman)), Tween 20, and LI 700®. Regulaid, for example, is a nonionic surfactant for use with plant growth regulators.

The agricultural composition may include a biocontrol agent. A “biocontrol agent” may include any agent applied to a plant including live microbial species such as bacteria, yeast, or fungus. The present inventors have discovered, in part, that the antimicrobial effect of the antimicrobial compositions described herein are restricted to Erwinia species such as E. amylovora and a few other bacterial species, but the compositions do not affect, for example, the growth of most non-pathogenic, flower-associated microorganisms. Thus, unlike traditional treatments such as streptomycin, the antimicrobial compositions described herein may be combined with biocontrol agents without significantly affecting the viability of the microbial species included in the biocontrol agent. Suitable biocontrol agents may include, without limitations any of the biocontrol agents listed in Table 1 or combinations thereof.

TABLE 1
Nonlimiting Examples of Biocontrol Agents

| Commercial | | Mode of | | |
|---|---|---|---|---|
| name | Active ingredients | action | Isolation source | Reference |
| Serenade Max | Bacillus subtilis | Antibiosis | Ubiquitous bacteria present | US-EPA Biopesticide |
| | strain QST 713 | | in soil, water and air | Registration |
| Double Nickel | Bacillus amyloliquefaciens | Antibiosis | Ubiquitous bacteria present | Certis USA |
| | strain D747 | | in soil, water and air | pesticide label |
| BlightBan C9-1 | Pantoea vagans C9-1 | Antibiosis | Stem tissue of apple in MI | (Ishimaru et al., 1988) |
| Bloomtime | Pantoea. agglomerans | Antibiosis | Gala′ apple blossoms | (Pusey, 1997) |
| Biological E325 | strain E325 | | near Wenatchee, WA | |
| BlighBan A506 | Pseudomonas fluorescens | Preemptive | Asymptomatic pear leaves | (Wilson and |
| | strain A506 | exclusion | near Healdsburg, CA | Lindow, 1992) |
| Blossom Protect | Aureobasidium pullulans | Preemptive | Apple leaves in Germany | Bio-ferm.com |
| | strain DSM 14941 | exclusion | | |
| | and 14942 | | | |

In some embodiments, the agricultural composition includes as a biocontrol agent Bacillus subtilis strain QST 713, Bacillus amyloliquefaciens strain D747, Pantoea vagans C9-1, Pantoea. agglomerans strain E325, Pseudomonas fluorescens strain A506, Aureobasidium pullulans strain DSM 14941 and 14942, or combinations thereof.

The agricultural composition may also include an additional active ingredient such as, without limitation, a fungicide, an herbicide, a biosanitizer product (such as Oxidate 2.0 from Biosafe 11c), a copper product (such as Cueva from Certis USA Ilc) or fertilizer.

The agricultural composition may include the antimicrobial composition at a concentration of 1 μM to 1 mM or any range therein. Suitably, the concentration of the antimicrobial composition in the agricultural composition is 10 μM to 300 μM.

In a further aspect of the present invention, methods for inhibiting the growth of an Erwinia species on a plant are provided. The methods may include contacting the plant with an effective amount of any one of the compositions described herein to inhibit the growth of the Erwinia species on the plant.

As used herein, a “plant” includes any portion of the plant including, without limitation, a whole plant or a portion of a plant such as a part of a root, leaf, stem, seed, pod, flower, cell, tissue plant germplasm, asexual propagate, or any progeny thereof. For example, an apple tree refers to whole the whole applie tree or portions thereof including, without limitation, the leaves, flowers, fruits, stems, roots, or otherwise. Suitable plants to be used in the present methods may include plants within the family Rosaceae. The Rosaceae are a family of flowering plants that produce many economically important edible fruits including, without limitation, apples, pears, quinces, apricots, plums, cherries, peaches, strawberries, and raspberries. Ornamental plants such as roses, firethorns, and meadowsweets are also within the family Rosaceae. In some embodiments, the plant within the family Rosaceae may be a plant producing edible fruits. In some embodiments, the plant within the family Rosaceae may be an ornamental plant. In some embodiments, the plant is an apple tree or a pear tree.

In some embodiments, the plant may be a plant capable of being infected with Erwinia amylovora and causing, for example, Fire Blight. Fire Blight is a contagious disease affecting apples, pears, and other members of the family Rosaceae.

As used herein, “contacting” may be carried out through any of the variety of procedures used to apply compositions to plants that will be apparent to the skilled artisan. Suitable application methods may include, without limitation spraying or dusting. Other suitable application procedures can be envisioned by those skilled in the art. Contacting may also be carried out indirectly via application, for example, to the soil surrounding a plant, trunk injection, or other plant media or substrates.

“Effective amount” is intended to mean an amount of a composition described herein sufficient to inhibit the growth of an Erwinia species on a plant by, for example, 10%, 20%, 50%, 75%, 80%, 90%, 95%, or 1-fold, 3-fold, 5-fold, 10-fold, 20-fold, or more compared to a negative control. In some embodiments, the effective amount of an antimicrobial composition either alone or in an agricultural composition may be 1 μM to 1 mM or any range therein. Suitably, the concentration of the antimicrobial composition either alone or in an agricultural composition is 10 μM to 300 μM. A “negative control” refers to a sample that serves as a reference for comparison to a test sample. For example, a test sample can be taken from a test condition including the presence of an antimicrobial composition and compared to negative control samples lacking the antimicrobial composition or including a composition not expected inhibit Erwinia growth (i.e., SEQ ID NO: 12 used in the Examples). One of skill in the art will recognize that controls can be designed for assessment of any number of parameters.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

Unless otherwise specified or indicated by context, the terms “a”, “an”, and “the” mean “one or more.” For example, “a protein” or “an RNA” should be interpreted to mean “one or more proteins” or “one or more RNAs,” respectively.

The following examples are meant only to be illustrative and are not meant as limitations on the scope of the invention or of the appended claims.

### EXAMPLES

### Example 1—Peptide Nucleic Acid (PNA) in Conjugation with Cell Penetrating Peptide (CPP) Causes Silencing of an Essential Gene in the Fire Blight Pathogen Erwinia amylovora and Inhibits Pathogen Growth

Erwinia amylovora is a Gram-negative bacterial plant pathogen in the family Enterobacteriaceae and is the causal agent of fire blight, a devastating disease of apple and pear. Fire blight is traditionally managed by the application of the antibiotic streptomycin during bloom, but this strategy has been recently challenged by the development and spread of streptomycin resistance. Thus, there is an urgent need for effective, specific, and sustainable control alternatives for fire blight. Antisense antimicrobials are oligomers of nucleic acid homologs with antisense sequence of essential genes in bacteria. The binding of these molecules to the mRNA of essential genes can result in translational repression and antimicrobial effect. Here, we explored the possibility of developing antisense antimicrobials against E. amylovora and using these compounds in fire blight control. We determined that a 10-nucleotide oligomer of peptide nucleic acid (PNA) targeting the start codon region of an essential gene acpP is able to cause complete growth inhibition of E. amylovora. See SEQ ID NO: 1. We found that conjugation of cell penetrating peptide (CPP) to PNA is essential for the antimicrobial effect, with CPP1 [(KFF)3K; SEQ ID NO: 2] being the most effective against E. amylovora. The minimal inhibitory concentration (MIC) of anti-acpP-CPP1 (2.5 μM; SEQ ID NO: 1 linked to SEQ ID NO: 2 via an ethylene glycol linker) is comparable to the MIC of streptomycin (2 μM). Examination of the antimicrobial mechanisms demonstrated that anti-acpP-CPP1 caused dose-dependent reduction of acpP mRNA in E. amylovora upon treatment and resulted in cell death (bactericidal effect). Anti-acpP-CPP1 (100 μM) is able to effectively limit the pathogen growth on stigmas of apple flowers, although less effective than streptomycin. Finally, unlike streptomycin that does not display any specificity in inhibiting pathogen growth, the antimicrobial effect of anti-acpP-CPP1 is restricted to E. amylovora and a few other bacterial species, but does not affect the growth of most non-pathogenic, flower-associated microorganisms. In summary, we demonstrated that PNA-CPP can cause an effective, specific antimicrobial effect against E. amylovora and may provide the basis for a novel approach for fire blight control.

### Introduction

Fire blight, caused by the bacterial pathogen Erwinia amylovora, is one of the most serious diseases of apple and pear in the United States and worldwide. Fire blight infection can occur in flowers, leaves, shoots, and fruits, resulting in yield reduction; the pathogen also can spread systemically through trees to the rootstock, ultimately resulting in tree death (Norelli et al., 2003; van der Zwet et al., 2012). Annual losses to fire blight and costs of control in the United States are estimated at over $100 million (Norelli et al., 2003).

The management of fire blight is challenged due to the availability of limited control options. As the pathogens enter plants through the natural opening of the flowers, antibiotic spray applications during bloom are the most effective and widely used control methods for fire blight (Norelli et al., 2003; Sundin et al., 2016). Streptomycin is the most effective antibiotic targeting E. amylovora, and has been used for fire blight management since the 1950s (Goodman, 1959). The intensive, long-term use of streptomycin, however, has resulted in the development of streptomycin resistance in the E. amylovora population. Since its original report in California in 1972 (Miller and Schroth, 1972), streptomycin resistance has been observed in most major apple-producing regions in the United States (Chiou and Jones, 1995; Evans, 2007; McGhee et al., 2011; Russo et al., 2008). In addition, agricultural application of streptomycin also raises significant concerns for the potential selection of antibiotic resistant bacteria in the environment, and the potential impact to human health (Sundin and Bender, 1996). Besides antibiotics, copper bactericides and other biological control products are also used for fire blight management, however the use of these materials is limited by their inconsistent control efficacy and copper use can also result in phytotoxicity (Johnson and Temple, 2013; Sundin et al., 2009; Tsiantos et al., 2003). Because of these reasons, developing effective control alternatives for fire blight has become an urgent need for sustainable apple and pear production in the United States (Khokhani et al., 2013; Sundin et al., 2016; Yang et al., 2014).

RNA silencing is the translational repression of an mRNA caused by the binding of an antisense RNA (Nakashima et al., 2012). In principle, the translation of any mRNA could be silenced by an antisense RNA with sequences complementary to the translational initiation sequences of the target mRNA (Bennett and Swayze, 2010; Good and Nielsen, 1998). The discovery of RNA silencing provides a powerful tool to artificially modulate gene expression. One application of RNA silencing is the synthesis of artificial RNA homologs to silence the expression of essential genes in microbes, and use of these compounds as antimicrobials (Rasmussen et al., 2007).

Antisense antimicrobials are short oligomers of nucleic acid homologs with antisense sequences to the translational initiation sites of essential genes of bacteria (Bai and Luo, 2012; Rasmussen et al., 2007). The binding of the antisense compounds to the translational initiation sites can lead to the silencing of these essential genes and subsequent growth inhibition (Bai and Luo, 2012). Compared to traditional antibiotics, antisense antimicrobials have many unique advantages including: 1. Unlike antibiotics that usually target a universal cellular process and kill bacteria with little selection, antisense antimicrobials can target a specific DNA sequence of the pathogen without affecting the survival of other potentially beneficial, environmental bacteria; 2. Unlike antibiotics that are typically limited to targeting a single cellular process, antisense antimicrobials can target any essential genes through sequence complementation, thus significantly enlarging the target selection; 3. In the case of a pathogen developing resistance to antisense antimicrobials through mutations, the resistance could be overcome by designing new antisense sequences against the mutated sequence.

The fact that DNA and RNA are unstable under UV and can be easily degraded by enzymes makes them undesirable materials for antisense antimicrobials. Improvements designed to modify or replace the sugar-phosphate backbone of DNA/RNA has resulted in nucleic acid homologs with significantly-enhanced stability (Bai and Luo, 2012). These nucleic acid homologs include peptide nucleic acids (PNAs), phosphorodiamidate morpholino oligomers (PMOs), and phosphorothioate oligonucleotides (PS-ODNs) (Bai and Luo, 2012). Among them, PNAs have shown promising antimicrobial effects against some animal pathogenic bacterial species (Good and Nielsen, 1998; Hatamoto et al., 2010; Kulyte et al., 2005; Kurupati et al., 2007; Nekhotiaeva et al., 2004). In PNA, the sugar-phosphate backbone of DNA/RNA was replaced with a pseudopeptide backbone, while nearly identical geometry and spacing of the bases was retained (Bai and Luo, 2012). This modification not only significantly enhances the stability, but also increases the PNA-RNA binding affinity, as RNA is negatively charged but PNA is electrically neutral (Bai and Luo, 2012). However, since PNA oligomers are large molecules, they may not enter cellular membranes as readily. Thus, the delivery of PNA oligomers into bacterial cells often requires external assistance. Cell penetrating peptides (CPPs) are short peptides of less than 30 amino acids that can penetrate cell membranes and deliver covalently conjugated cargoes into cells (Abes et al., 2007; Bai and Luo, 2012). Conjugation of CPPs with PNAs has been demonstrated to significantly increase PNA entry into bacterial cells (Rasmussen et al., 2007; Zatsepin et al., 2005). Two features of CPPs that are required for cell penetrating ability include amphipathicity and positive charge (Bai and Luo, 2012; Wu et al., 2007). These characteristics are acquired by synthesizing CPPs with an alteration of cationic amino acid residues and nonpolar residues.

PNA-CPPs have been successfully used as antisense antimicrobials in both in vitro and in vivo trials against animal pathogenic bacteria. For example, CPP-conjugated PNAs targeting essential genes such as acpP, inhA, gyrA, ompA, 16 s rRNA, and adk have shown significant growth inhibition effect against a number of bacteria including Escherichia coli (Good and Nielsen, 1998; Tan et al., 2005), Pseudomonas aeruginosa (Ghosal and Nielsen, 2012), Staphylococcus aureus (Hatamoto et al., 2010), Mycobacterium smegmatis (Kulyte et al., 2005), and Klebsiella pneumoniae (Kurupati et al., 2007). Previous research suggests that the start codon and Shine-Dalgarno (SD) region of the mRNA are the most sensitive sequences for inhibition caused by antisense antimicrobials (Dryselius et al., 2003).

Although PNA-CPPs have shown some promising applications in controlling bacterial infections in animal models, to our knowledge, no research has explored the use of PNA-CPP in controlling plant diseases. We previously have identified regulatory small RNAs in the fire blight pathogen E. amylovora and have described the roles of these sRNAs in regulating various virulence and cellular functions (Zeng et al., 2013; Zeng and Sundin, 2014). These findings suggest that RNA silencing is a naturally occurring process in the E. amylovora and that it is possible that the expression of a given gene could also be modulated by artificially synthesized RNA homologs. In this research, we explored the proof-of-concept of using PNA-CPP that targets an essential gene acpP in E. amylovora as an antimicrobial, and using this compound to control fire blight.

### Material and Methods

### Bacterial Strains, Culture Conditions and PNA-CPP Synthesis.

The highly-virulent strain E. amylovora Ea110, which was isolated from an apple orchard in Michigan (Zhao et al., 2005), was used in this study. Bacterial strains were stored at −80° C. in 15% glycerol and cultured in Luria-Bertani (LB) medium at 28° C. PNA-CPP was synthesized using Bts oligomerization method by Panagene Inc (Daejeon, Korea). Streptomycin and PNA-CPP were implemented at rates indicated in each assay.

### Measurement of Bacterial Growth Inhibition

Erwinia amylovora Ea110 was cultured in LB broth overnight and then cell concentrations were adjusted to 5×10⁵ CFU/ml in LB broth. A total of 80 μl of the bacterial suspension in LB was added into each well of a 96 well plate. Lyophilized PNA-CPP was resuspended in water to a stock concentration of 100 μM and was serial diluted. Twenty μl of the diluted PNA-CPP solution was added into each well that contained 80 μl of the bacterial suspension in LB to make the final concentrations indicated in each assay. Water and streptomycin were added to the wells as negative and positive controls. The plate was incubated at 28° C. with orbital shaking in a BioTek Synergy H1 microplate reader (BioTek, Winooski, Vt., USA) for 20 hr. During the incubation, the OD 600 of each well was measured every 10 min. The lowest concentration of a PNA-CPP that prevented growth after 20 hr represented the MIC. Three replicates were included in each testing, and the experiment was repeated twice.

### Viability Test

Overnight cultures of E. amylovora Ea110 were adjusted to 5×10⁵ CFU/ml in LB broth. Forty microliters of anti-acpP-CPP1 (100 μM), streptomycin (100 μM), or H₂O were added into 460 μl of the LB broth containing E. amylovora cells to reach a final concentration of 8 μM of the compounds mentioned above. The cultures were incubated at 28° C. with 200 rpm of continuous shaking. Samples were taken at 0, 1, 2, 3, 4, 5, and 6 hours post inoculation. Bacterial cells from each sample were collected by centrifugation (6500 rpm for 8 min), washed with sterile water to remove the residual compounds, diluted 10² to 10⁵-fold, and plated on LB agar plates. Colonies formed after 48 hr-incubation at 28° C. are counted and original cell concentration was calculated. Five replicates were included in each assay and the experiment was repeated twice with similar results observed.

### Fluorescence Microscopy

Bacterial cells were washed with sterile water to remove residual medium from the culture. LIVE/DEAD BacLight bacterial viability kit (Molecular Probes, Eugene, Oreg., USA) components A and B were added to the cells following the manufacturer's instructions. Green fluorescence and red fluorescence of cells stained were observed using a Zeiss Scope A1 fluorescence microscope equipped with a FITC filter and a DAPI filter (Oberkochen, Germany). Images were acquired with a Spot RT3 camera using the Spot Advanced software (Sterling Heights, Mich., USA).

### RNA Isolation

E. amylovora Ea110 was inoculated into LB broth at the final concentration of 5×10⁵ CFU/ml, and was treated with various concentrations of PNA-CPP, water, or streptomycin. The inoculated cells were incubated at 28° C. with orbital shaking, and were collected for RNA isolation at 15.5 hrs post inoculation. Total bacterial RNA was isolated using the RNeasy Protect Bacteria Mini Kit (Qiagen, Valencia, Calif., USA) following the manufacturer's instructions. The quality and quantity of RNA was measured with a Nanodrop 2000c (Thermo Scientific, Wilmington, Del., USA).

### Northern Blot

The probe for acpP detection in Northern blots was synthesized from polymerase chain reaction (PCR) using acpP primers (forward primer 5′-TGG GCG TTA AGC AGG AAG AAG-3′ (SEQ ID NO: 5) and reverse primer 5′-TAC GCC TGG TGA CCA TTG AT (SEQ ID NO: 6)). The PCR product was purified using a QIAquick PCR Purification kit (Qiagen) and labelled using a Biotin DecaLabel DNA Labeling Kit (Thermo Fisher Scientific, Grand Island, N.Y.) as per instruction of kit manual. Thirty micrograms of RNA was loaded for each sample and was separated on a formaldehyde denaturing gel in MOPS buffer. BrightStar®-Plus positively-charged nylon membranes (Thermo Fisher Scientific) were used for transfer of RNA from gel to membrane. Transfer, pre-hybridization, and hybridization were performed following the manufacturer's instructions of the Northern Max kit (Thermo Fisher Scientific). The biotin signal was detected using a Biotin Chromogenic Detection Kit (Thermo Fisher Scientific).

### Detached Apple Flower Assay

Freshly opened flowers were collected from apple trees Malus×domestica ‘McIntosh’ from the Connecticut Agricultural Experiment Station Lockwood farm in Hamden, Conn., USA) in May 2016. The pedicel of the flowers was detached from the flower cluster, and each flower was placed in a 7 ml plastic tube containing a 10% sucrose solution through a hole created in the plastic cap. One microliter of E. amylovora (Ea110) at the concentration of 10⁷ CFU/ml was inoculated onto the five stigmas of each flower (approximately 0.2 μl per stigma). Twenty microliters of the PNA-CPP at different concentrations were evenly applied onto the stigmas of each flower 2 hr before and 19 hr after the inoculation. Water and streptomycin (100 μM) were used as negative and positive controls. In the PNA-bacteria mix treatment, PNA at the concentration 20 μM was mixed with an equal volume of E. amylovora cells at the concentration of 10⁷ CFU/ml in a microcentrifuge tube and incubated for 30 min at 22-25° C. Two microliters of the mixture were applied evenly onto the five stigmas of the flowers. The pathogen population on the apple stigmas was quantified at 42 hr post inoculation, by dissecting the stigmas from the flowers, and suspend them in 1 ml of 0.5×PBS. A Taqman probe realtime PCR assay was used to quantify the pathogen amount. Primers used in this assay are: amsk120F: 5′-CAT GCA ATT TCC AGT TTC CT-3′ (SEQ ID NO: 7); amsk120R: 5′-GCA TGA CGG TTA ACC AAA TC-3′ (SEQ ID NO: 8), amsk120Probe: 5′-TGC GTG ACC TGA TTC AGC ACA A-3′ (SEQ ID NO: 9). The reaction was performed on a Biorad CFX96 Realtime PCR machine. The cell concentrations were calculated by f (Cq)=−3.47 Cq+45.513. A standard curve was generated using five predetermined concentrations of Ea110 (10⁷, 10⁶, 10⁵, 10⁴, 10³ CFU/ml) cells. Six flowers were used in each treatment and the experiment was repeated twice with similar results observed. The box plot was generated by R (R Core Team, 2015), and statistical analysis was performed using one-way ANOVA model in the ‘stats’ package in R. Letters above the bars denote the level of significance (α=0.05).

### Collection of Apple Flower-Associated Bacteria

Fifty flowers were collected from apple trees Malus×domestica ‘McIntosh’ from the Hamden, Conn. Lockwood farm in May 2016. The stigmas from all 50 flowers were removed, placed into 3 ml of 0.5×PBS, vortexed for 20 seconds, and sonicated for 5 min in a water bath sonicator to release the stigma associated bacteria. PBS buffer containing bacteria cells was serially diluted (10×, 100× and 1000×) and plated on LB agar medium and incubated at 28° C. for 48 h. On the basis of morphological colony characteristics, 200 bacterial colonies were shortlisted, sub-cultured, and identified using 16s rDNA sequencing using the forward primer 63f (5′-CAG GCC TAA CAC ATG CAA GTC-3′) (SEQ ID NO: 10) and reverse primer 1387r (5′-GGG CGG WGT GTA CAA GGC-3′) (SEQ ID NO: 11). Fifteen different species were selected from the 200 bacterial cultures for testing against anti-acpP-CPP1 and streptomycin. Biological control agents Pseudomonas fluorescence A506, Bacillus amyloliquefaciens D747 and Pantoea agglomerans E325 were isolated from the original products on LB agar plate.

### Results

PNAs with Antisense Sequence to the Start Codon Region of acpP Caused Growth Inhibition of E. amylovora when Conjugated with CPP.

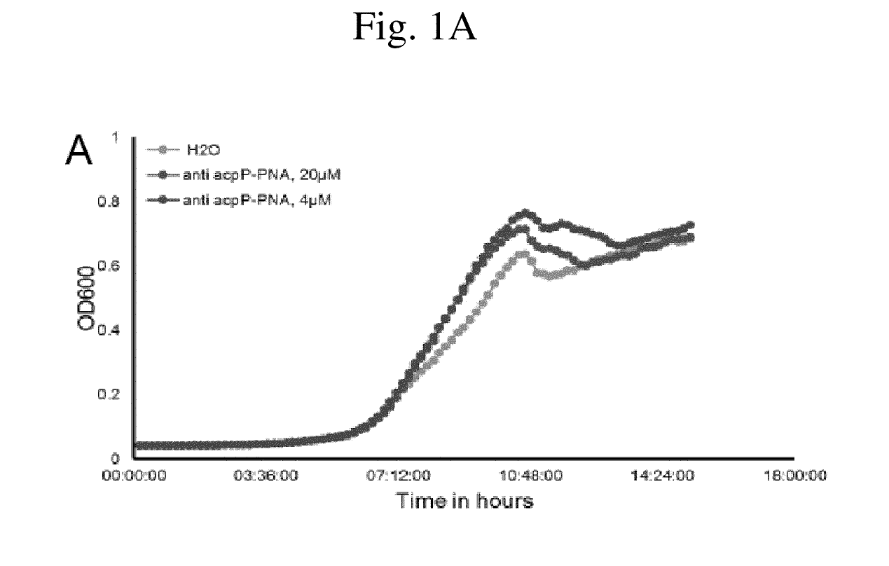

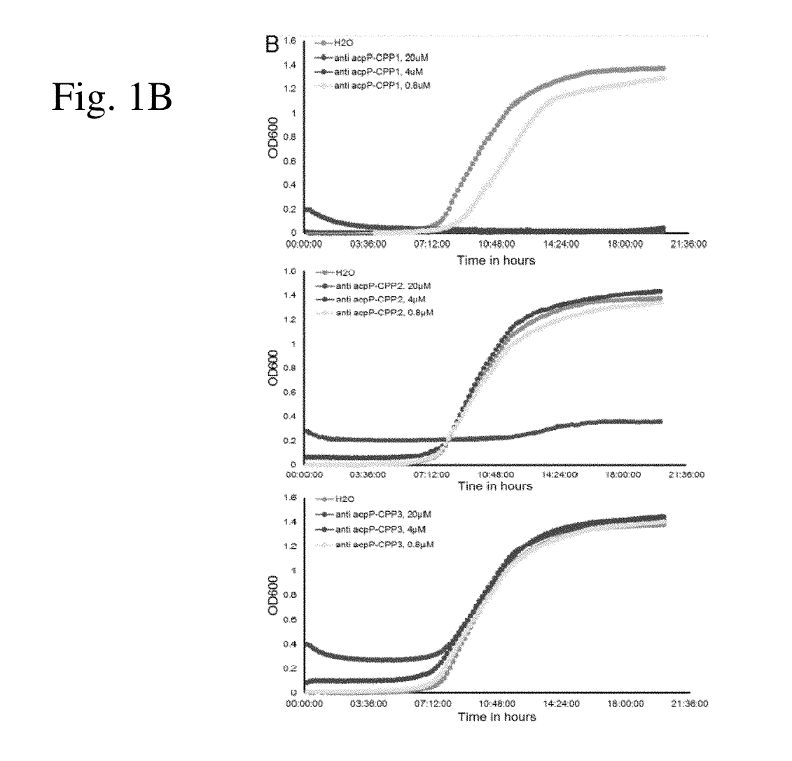

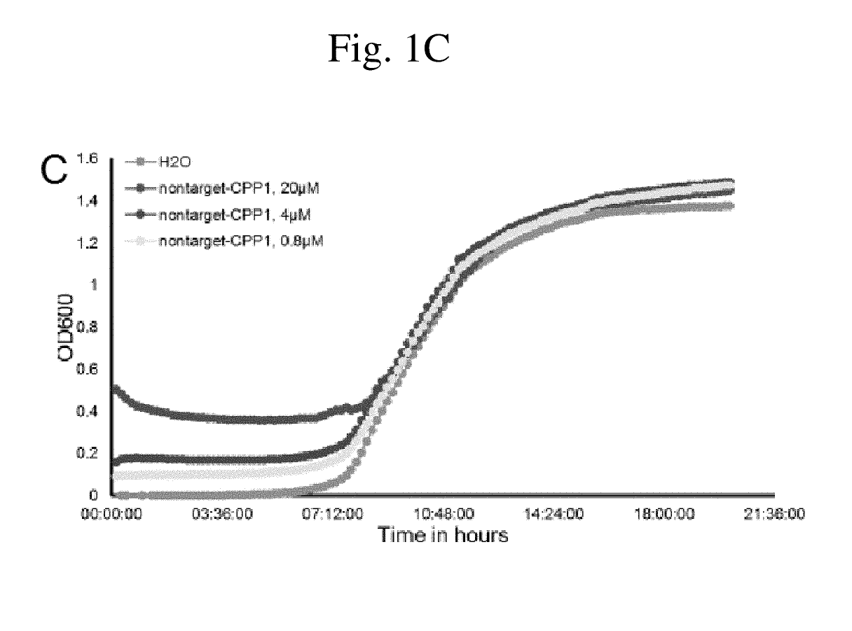

To explore the possibility of using PNAs as antimicrobials against the fire blight pathogen E. amylovora, we first synthesized a 10-nucleotide PNA oligomer with antisense sequence to the start codon region (−5 to +5) of a previously discovered essential gene acpP (encoding an acyl carrier protein), anti-acpP PNA (Table 2) (Zhang and Cronan, 1998). The effect of the anti-acpP PNA on the growth of E. amylovora was evaluated under an in vitro condition. Compared to the water-treated cells, cells treated with anti-acpP PNA did not display any growth inhibition at either concentration tested (FIG. 1A).

TABLE 2
PNA-CPP molecules used in this Example

| Compound name | Sequence |
|---|---|
| Anti-acpP PNA | 5′-ctcatactat-3′ (SEQ ID NO: 1) |
| Anti-acpP-CPP1 | (KFF)3K-egl-ctcatactat-3′ (SEQ ID NO: 2 and 1; |
| | egl = ethylene glycol linker) |
| Anti-acpP-CPP2 | 5′-ctcatactat-BX(RXR)4 (SEQ ID NO: 1 and 3) |
| Anti-acpP-CPP3 | YARVRRRGPRGYARVRRRGPRRC-ctcatactat-3′ (SEQ |
| | ID NO: 4 and 1) |
| Nontarget PNA- | (KFF)3K-egl-accatggtgg-3′ (SEQ ID NO: 2 and 12 |
| CPP1 | with ethylene glycol linker) |

Bold letters indicate PNA sequence, in the 5′ to 3′ order.

As the ineffectiveness of the anti-acpP PNA on E. amylovora growth could be due to the inefficient entry of PNA into the cytoplasm, we determined whether conjugation of the anti-acpP PNA with CPP would result in enhanced antimicrobial effect. Three formulations of CPP (CPP1, CPP2, and CPP3) that were demonstrated to have promising delivery efficiency of PNAs in animal pathogenic bacteria were then individually conjugated to the anti-acpP PNA, resulting in anti-acpP-CPP1, anti-acpP-CPP2, and anti-acpP-CPP3 (Table 2). The effect of the CPP-conjugated PNAs on E. amylovora growth was tested under the same conditions. Compared to the water control, addition of anti-acpP-CPP1 or anti-acpP-CPP2 both resulted in complete growth inhibition of E. amylovora at the 20 μM concentration, whereas addition of anti-acpP-CPP3 did not cause any growth inhibition at any concentrations (FIG. 1B). The growth inhibition caused by anti-acpP-CPP1 was more potent than the inhibition caused by anti-acpP-CPP2, suggesting that CPP1 [(KFF)3K] was the most efficient CPP in delivering PNA into E. amylovora. To determine if the growth inhibition caused by PNA-CPP was through specific targeting of acpP, a PNA with random nucleotide sequence in conjugation with CPP1 (nontarget PNA-CPP1) was also tested (Table 2). The nontarget-CPP1 did not cause any inhibition of E. amylovora growth (FIG. 1C).

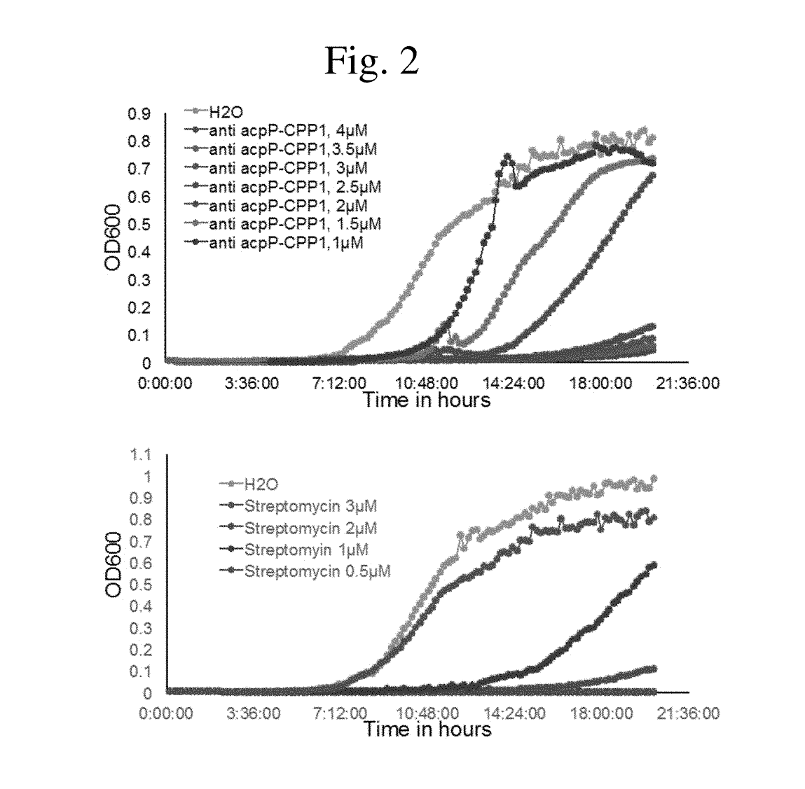

We further determined that the minimal inhibitory concentration (MIC) of anti-acpP-CPP1 and streptomycin was 2.5 μM and 2 μM, respectively (FIG. 2). In addition, we showed that the growth inhibition caused by anti-acpP-CPP1 is positively correlated with the concentrations of anti-acpP-CPP1. Taken together, these results suggest that CPP-conjugated PNA with antisense sequence targeting the start codon of acpP is able to cause potent growth inhibition of E. amylovora under in vitro conditions. This inhibition was caused by the antisense sequence of acpP on PNA. CPP was essential in delivering PNA into E. amylovora and causing the antimicrobial effect. Among the three CPPs previously used in animal pathogens, CPP1 was the most efficient for PNA delivery into E. amylovora.

E. amylovora Cells Treated with Anti-acpP-CPP1 Showed Reduced acpP mRNA Levels.

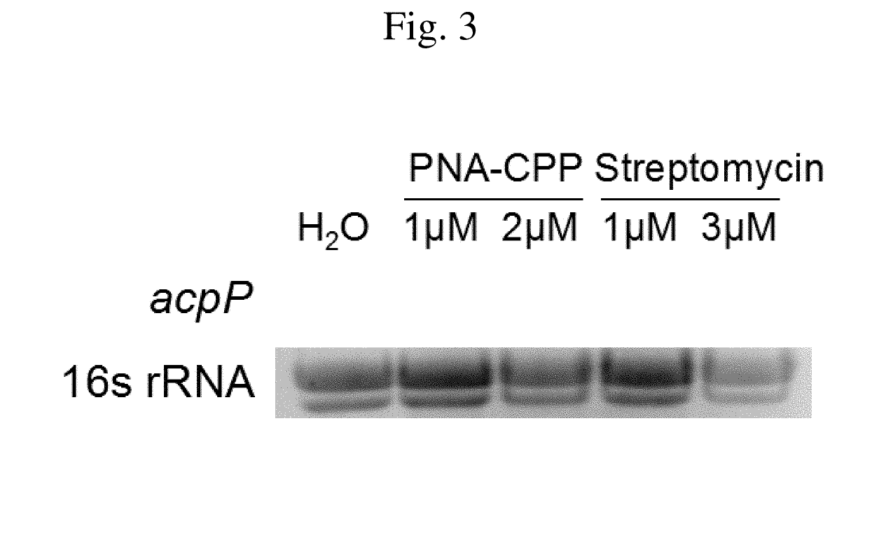

After entering the bacterial cytoplasm, PNA-CPP is believed to bind to the start codon region of the target mRNA and block its translation (Good and Nielsen, 1998; Wang and Xu, 2004). However, whether the PNA-mRNA binding would also cause the degradation of the target mRNA is not clear. We compared the levels of acpP mRNA in E. amylovora cells treated with water, anti-acpP-CPP1 at two sub-lethal concentrations (1 μM and 2 μM), and streptomycin at sub-lethal concentrations (1 μM and 3 μM) using Northern blot. Compared to the water-treated cells, cells treated with anti-acpP-CPP1 showed a visual reduction in the level of acpP mRNA. This reduction is positively correlated with anti-acpP-CPP1 concentrations (FIG. 3). In contrast, streptomycin treatment did not affect the acpP mRNA abundance. These observations suggest that anti-acpP-CPP1 binding to acpP mRNA caused the degradation of acpP mRNA. It also indicates that the growth inhibition caused by anti-acpP-CPP1 is through a gene specific interaction with acpP mRNA.

The Growth Inhibition of E. amylovora by Anti-acpP-CPP1 is Bactericidal.

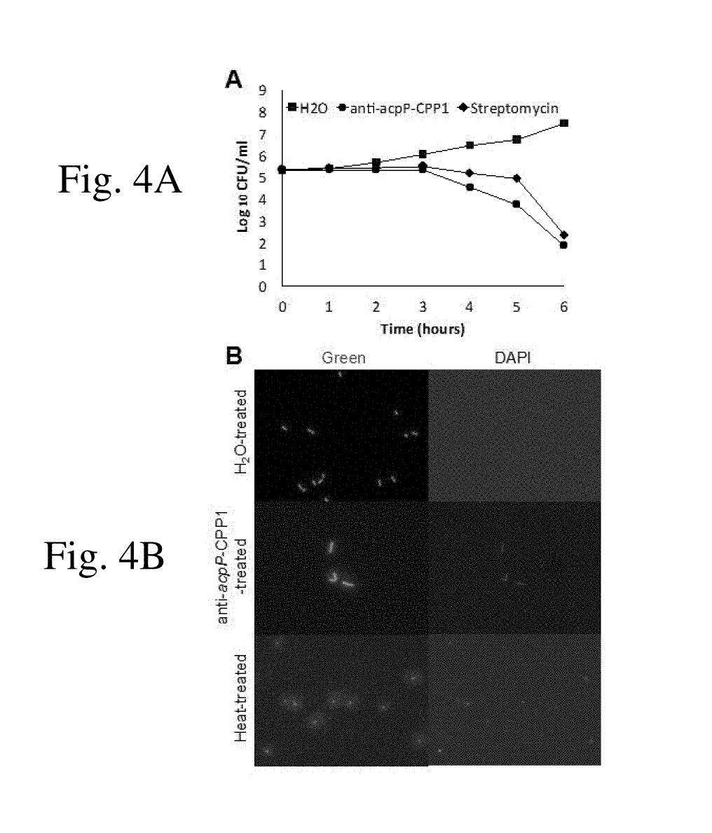

To determine whether the growth inhibition of E. amylovora by anti-acpP-CPP1 was bactericidal or bacteriostatic, we tested the viability of E. amylovora cells in LB broth at different time points after treatment of anti-acpP-CPP1 (8 μM), streptomycin (8 μM), or water, using a serial dilution plating method. In the water-treated sample, we observed an exponential increase in the amount of viable cells after inoculation (FIG. 4A). Following treated with anti-acpP-CPP1, the viable cell count remained the same during the first 3 hr, displaying a bacteriostatic effect (FIG. 4A). After 3 hr, the viability of cells treated with anti-acpP-CPP1 drastically declined, which suggested that anti-acpP-CPP1 caused a bactericidal effect during this period. After 6 hr of treatment with anti-acpP-CPP1, the number of viable cells was reduced by more than 1000-fold (log₁₀ CFU/ml reduced from 5.3 at 0 hr to 1.8 at 6 hr, FIG. 4A), at a slightly faster rate than streptomycin. These observations suggest that the growth inhibition of E. amylovora caused by anti-acpP-CPP1 is bactericidal, similar to the traditional antibiotic streptomycin.

The mechanism of the growth inhibition caused by anti-acpP-CPP1 was also studied using a bacterial viability kit and fluorescence microscopy. At 6 hr after inoculation, cells treated with water were green fluorescent but not red fluorescent when stained with a mixture of fluorescent dye SYTO 9 and propidium iodide, suggesting that these cells were viable (FIG. 4B). However, most cells treated with anti-acpP-CPP1 at a concentration above the MIC (8 μM) were both green fluorescent and red fluorescent, suggesting that these cells lost viability (FIG. 4B). Interestingly, cells treated with anti-acpP-CPP1 showed an elongated cell shape compared to the water-treated cells (FIG. 4B). No cell lysis (as shown in the heat-treated cells in FIG. 4B) was observed following treatment with anti-acpP-CPP1, which suggests that the bactericidal effect may not be caused by physical disintegration of the cell structure (FIG. 4B). The fluorescence microscopy observation is consistent with the plating method, together suggesting that the growth inhibition of E. amylovora caused by anti-acpP-CPP1 is bactericidal.

### Anti-acpP-CPP1 was Able to Inhibit Pathogen Growth on Apple Stigmas.

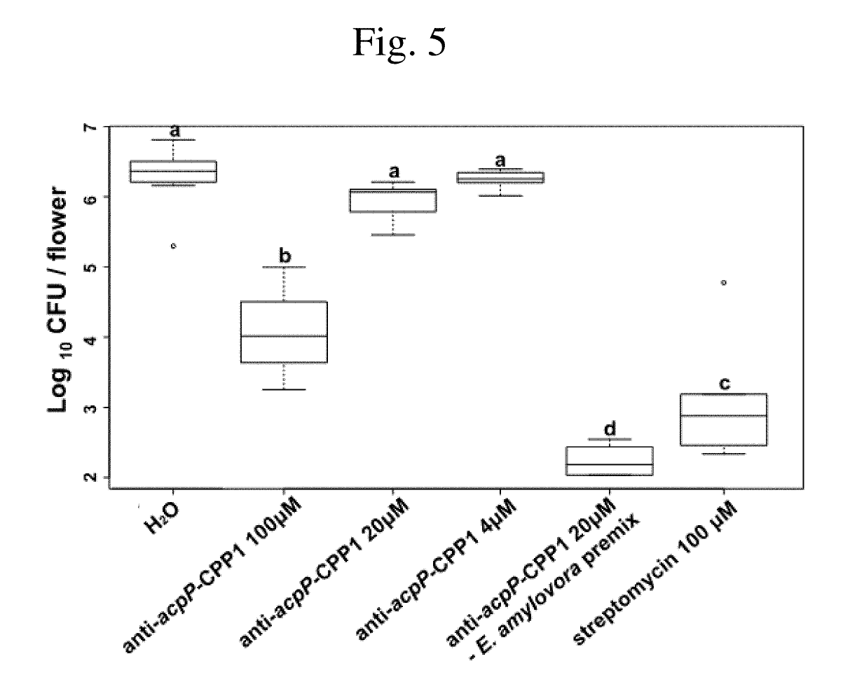

Following demonstration that anti-acpP-CPP1 has potent growth inhibition of E. amylovora under in vitro conditions, we further evaluated the effectiveness of anti-acpP-CPP1 in inhibiting E. amylovora growth on apple stigmas using a detached flower assay. Compared to the water-treated control, stigmas treated with 100 μM anti-acpP-CPP1 showed a significant reduction of E. amylovora population (>100-fold reduction, FIG. 5). However, this reduction was less potent in comparison to the reduction by streptomycin (FIG. 5). At lower concentrations, anti-acpP-CPP1 treatment did not result in significant reduction of the pathogen population when directly applied to the apple stigmas (FIG. 5). However, premixing anti-acpP-CPP1 (20 μM) with E. amylovora cells for 30 min before applying the cell-compound mixture onto the apple stigmas resulted in excellent reductions of pathogen populations on apple stigmas (FIG. 5). The results above suggest that anti-acpP-CPP1 is able to inhibit pathogen growth on apple stigmas when applied at 100 μM. It also suggests that the efficacy of anti-acpP-CPP1 may be improved if optimal cell-compound contact can be established.

Effect of Anti-acpP-CPP1 on Microbiome Associated with Apple Flowers.

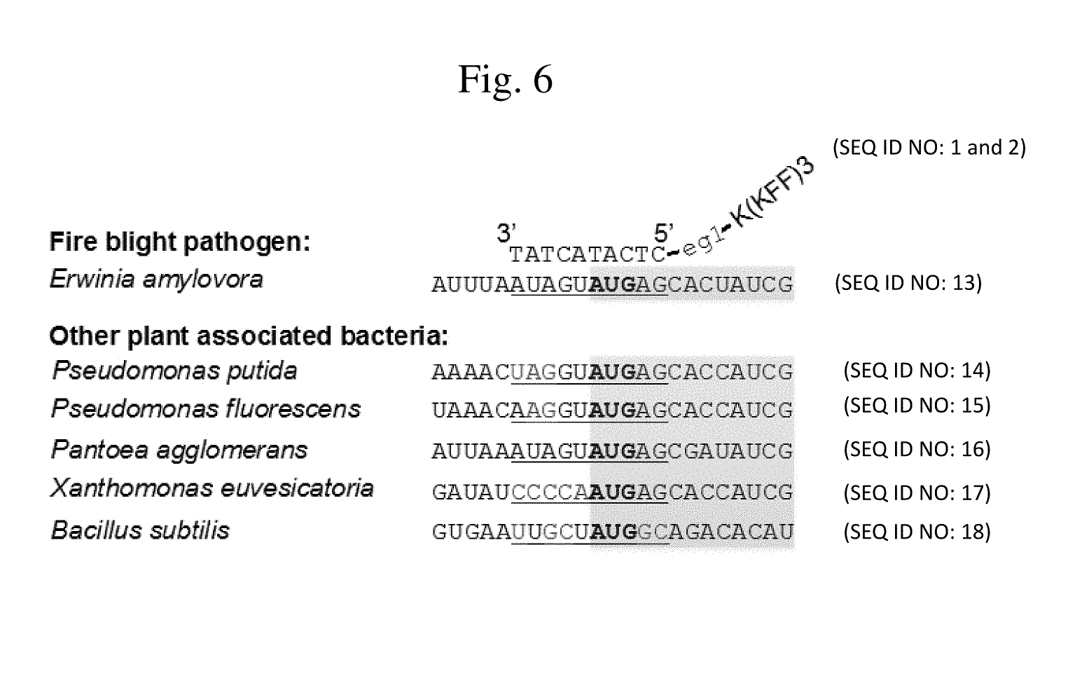

Compared to the traditional antibiotics that kill microbes with little selection, antisense antimicrobials inhibit bacterial growth through a sequence specific manner. To determine whether anti-acpP-CPP1 affects the growth of environmental microbes associated with apple flowers, bacteria were first cultured from freshly collected ‘McIntosh’ apple flowers. Two hundred bacterial colonies were subcultured, and the identities of these colonies were determined by 16S rDNA sequencing. The sequencing and Blast analyses suggest that these bacteria belong to 15 different species (Table 3). Representative strains from each species were tested for their susceptibility to anti-acpP-CPP1 and streptomycin at concentrations above the MICs for E. amylovora (4 μM and 20 μM, respectively). Our results suggest that anti-acpP-CPP1 did not affect the growth of most species (12/15), such as Pseudomonas sbsp., Bacillus muralis, Curtobacterium plantarum, and multiple species in the Pantoea genus. However, three species, P. ananatis, P. agglomerans, and Lysobacter oligotrophicus displayed susceptibility to anti-acpP-CPP1 similar to E. amylovora. Comparison of the start codon sequences of acpP in species with genomes available in NCBI revealed that most species contain sequence discrepancies with E. amylovora in this region (FIG. 6). P. agglomerans that has an identical sequence as E. amylovora was also susceptible to anti-acpP-CPP1. In addition to the bacterial species isolated from the apple flower, we also tested the susceptibility of three microorganisms from fire blight biological control products to anti-acpP-CPP1. Similar to the environmental isolates, Pseudomonas fluorescens A506 and Bacillus amyloliquefaciens D747 (active ingredients of BlightBan A506 and Double Nickel) were not affected by anti-acpP-CPP1 whereas P. agglomerans E325 (active ingredient of Bloomtime Biological) was susceptible (Table 3). All strains tested are susceptible to streptomycin with the exception of P. fluorescens A506 (Table 3).

TABLE 3
The effect of anti-acpP-CPPl and streptomycin

| | Growth in LB | Growth in LB | |
|---|---|---|---|
| | broth amended | broth amended | |
| | with | with | Growth |
| | anti-acpP- | streptomycin | in LB |
| Bacterial species | CPP1 (4 μM) | (20 μM) | broth |
| Environmental isolates | | | |
| recovered from apple | | | |
| flowers* | | | |
| Pseudomonas savastanoi | + | − | + |
| Pseudomonas syringae | + | − | + |
| Pseudomonas marginalis | + | − | + |
| Pseudomonas trivialis | + | − | + |
| Pseudomonas rhizosphaerae | + | − | + |
| Pseudomonas graminis | + | − | + |
| Pseudomonas simiae | + | − | + |
| Pseudomonas poae | + | − | + |
| Bacillus muralis | + | − | + |
| Pantoea ananatis | − | − | + |
| Pantoea brenneri | + | − | + |
| Pantoea vegans | + | − | + |
| Pantoea agglomerans | − | − | + |
| Curtobacterium plantarum | + | − | + |
| Lysobacter oligotrophicus | − | − | + |
| Bacterial species from | | | |
| commercial biocontrol | | | |
| agents for fire blight | | | |
| Pantoea agglomerans E325 | − | − | + |
| (Bloomtime Biological) | | | |
| Pseudomonas fluorescence | + | + | + |
| A506 (BlightBan A506) | | | |
| Bacillus amyloliquefaciens | + | − | + |
| D747 (Double Nickel) | | | |

*Bacterial species were identified by 16S rDNA sequencing. The names of the species are the ones with the highest similarity from the NCBI blast search.

### DISCUSSION

In this study, we demonstrated the proof-of-concept of using PNA-CPPs as antimicrobials against the fire blight pathogen E. amylovora. We showed that a 10-bp oligomer of PNA containing antisense sequence of the essential gene acpP is able to cause complete growth inhibition of E. amylovora at a similar efficacy of streptomycin under in vivo conditions. We also showed that conjugation of the PNA with a CPP is essential for the PNA to exert antimicrobial effect in E. amylovora and identified the most effective CPP sequence for E. amylovora. We provided evidence that the antimicrobial effect observed is bactericidal. And finally, no phytotoxicity was observed during the flower assay. These qualities suggest that PNA-CPPs are good alternatives for streptomycin in future fire blight management. Conditions generated from this research will add to our knowledge base for the practical development of formulations, rates, and timing for future applications.

Considerations that are often taken into account when developing antimicrobials include effectiveness, specificity, sustainability, and cost. We demonstrated that anti-acpP-CPP1 has a potent antimicrobial effect against E. amylovora with high strain specificity. We hypothesize that the antisense antimicrobials may also have good sustainability: if resistance is developed through mutations, the mutated nucleotide(s) could be identified by sequencing, and new antisense molecules could be synthesized to target the mutated sequence. Some bacteria may be able to generate resistance by acquiring multi-drug transporters. However, it is not very likely in this case as previous work demonstrated that PNA-CPPs cannot be readily transported out of the cells due to the large molecular weight (Nikravesh et al., 2007).

Prior to this research, the use of PNA-CPPs in controlling bacterial infections has been explored in human medicine. Compared to human medicine application, there are essential differences in agricultural use. First, the delivery method and locations where the antimicrobial activity occurs are different in the two systems. Antimicrobials for human medical purposes are usually delivered through the circulation system and the antimicrobial activity occurs mostly internally in human body. For agricultural use, the delivery is mostly achieved through aerial spray to the plant surface, and the antimicrobial activity occurs mostly externally on the plant surface. Second, non-pathogenic, beneficial bacteria are not readily used as a disease control strategy in human medicine, but the beneficial, pathogen-antagonistic microbes are of great use in plant disease management as biological controls. This emphasizes a need of selectively targeting the pathogen without affecting the non-pathogenic, beneficial bacteria in agricultural settings.

With the differences between the two systems, our study demonstrated that the application of a PNA-CPP can reduce the bacterial pathogen population on plant surfaces. Our results also emphasized the importance of ensuring the compound-bacteria contact for the optimization of antimicrobial results, as we showed that the premixing bacteria with PNA-CPP ensuring the full bacteria-compound contact had the most potent antimicrobial effect. It is possible that the use of a nonionic surfactant may be able to improve the antimicrobial effect of PNA-CPP on plant surfaces. In addition, whether the reduction in pathogen population on stigmas would also result in the reduction in blossom blight infection incidence needs to be further evaluated in future orchard trials. Many new challenges in formulation chemistry, product rates, and timing of sprays must be met.

Our study also showed that the antimicrobial effect of PNA-CPP molecules is specific to E. amylovora but not to many other environmental microorganisms. Not affecting the environmental microbiome has many benefits. First, healthy microbiota serves as competitors of the pathogens for nutrients, space and can have a positive effect in preventing and limiting disease occurrence. Second, the selective pressure on the total microbiome of antibiotics often facilitated the development and spread of antibiotic resistance into pathogen population (Sundin and Bender, 1996). For example, the streptomycin resistance genes strA-strB that confer streptomycin resistance in the fire blight pathogen E. amylovora are believed to be acquired from saprophytic bacteria isolates such as P. agglomerans through horizontal gene transfer (Chiou and Jones, 1991). Thus, compared to streptomycin and other antibiotics, PNA-CPP may be a more sustainable management approach for fire blight.

One strategy of reducing the usage of antibiotics in tree fruit disease management is to combine biocontrol agents with antibiotics. However, as antibiotics and biocontrol agents are often non-compatible, the antibiotics and biocontrol agents often have to be applied separately, which not only adds labor cost and time constraints, but also may reduce the efficiency. Here we showed that anti-acpP-CPP1 is compatible with multiple biocontrol agents such as P. fluorescens and B. subtilis. The combined application of biological control agents with PNA-CPP may further enhance the control efficacy.

We observed that PNA-CPP antisense antimicrobials caused degradation of the target mRNA of an essential gene with a subsequent bactericidal effect against E. amylovora. This suggests that the antimicrobial effect of PNA-CPP on E. amylovora is potent and permanent. It also suggests that besides the potential application in disease management, PNA-CPPs may also be used as an effective approach to modulate gene expression in E. amylovora and potentially other bacteria. As the mRNA inhibition and dose of PNA-CPP is positively correlated, gene expression can be easily repressed at different levels for molecular research purposes. Thus, the gene expression repression by PNA-CPPs may have potential advantages over the gene knockout approach.

In summary, we performed the first exploration of using PNA-CPPs in controlling a bacterial plant disease. Plant diseases caused by bacteria have a long history of control difficulties, and the lack of effective management options results in significant economical losses worldwide (Sundin et al., 2016). The results produced from this work suggest that antisense antimicrobials may be a valuable future choice for bacterial plant disease management.

### REFERENCES

Abes, R., Arzumanov, A. A., Moulton, H. M., Abes, S., Ivanova, G. D., Iversen, P. L., Gait, M. J., and Lebleu, B. (2007). Cell-penetrating-peptide-based delivery of oligonucleotides: an overview. Biochem. Soc. Trans. 35, 775-779.
Bai, H., and Luo, X. (2012). “Antisense antibacterials: from proof-of-concept to therapeutic perspectives.” in A Search for Antibacterial Agents, ed. V. Bobbarala (INTECH Open Access Publisher), 319-344.
Bennett, C. F., and Swayze, E. E. (2010). RNA targeting therapeutics: molecular mechanisms of antisense oligonucleotides as a therapeutic platform. Annu. Rev. Pharmacol. Toxicol. 50, 259-293.
Chiou, C. S., and Jones, A. L. (1991). The analysis of plasmid-mediated streptomycin resistance in Erwinia amylovora. Phytopathology 81, 710-714.
Chiou, C. S., and Jones, A. L. (1995). Expression and identification of the strA-strB gene pair from streptomycin-resistant Erwinia amylovora. Gene 152, 47-51.
Dryselius, R., Aswasti, S. K., Rajarao, G. K., Nielsen, P. E., and Good, L. (2003). The translation start codon region is sensitive to antisense PNA inhibition in Escherichia coli. Oligonucleotides 13, 427-433.
Evans, C. K. (2007). Survey results of Erwinia amylovora in Utah for resistance to streptomycin and investigations comparing kasugamycin (kasumin) to streptomycin and oxytetracycline for control of fire blight. Acta Hortic. 793, 433-437.
Ghosal, A., and Nielsen, P. E. (2012). Potent antibacterial antisense peptide-peptide nucleic acid conjugates against Pseudomonas aeruginosa. Nucleic Acid Ther. 22, 323-334.
Good, L., and Nielsen, P. E. (1998). Inhibition of translation and bacterial growth by peptide nucleic acid targeted to ribosomal RNA. Proc. Natl. Acad. Sci. U.S.A 95, 2073-2076.
Goodman, R. N. (1959). “The influence of antibiotics on plants and plant disease control.” in Antibiotics: Their Chemistry and Non-Medical Uses., ed. H. S. Goldberg (Princeton, N. J.: D. van Nostrand and Company, Inc.), 322-448.
Hatamoto, M., Ohashi, A., and Imachi, H. (2010). Peptide nucleic acids (PNAs) antisense effect to bacterial growth and their application potentiality in biotechnology. Appl. Microbiol. Biotechnol. 86, 397-402.
Johnson, K. B., and Temple, T. N. (2013) Evaluation of strategies for fire blight control in organic pome fruit without antibiotics. Plant Dis. 97, 402-409.
Khokhani, D., Zhang, C., Li, Y., Wang, Q., Zeng, Q., Yamazaki, A., Hutchins, W., Zhou, S.-S.,
Chen, X., and Yang C.-H. (2013). Discovery of plant phenolic compounds that act as type three secretion system inhibitors or inducers of fire blight pathogen Erwinia amylovora. Appl. Environ. Microbiol. 79, 5424-5436.
Kulyte, A., Nekhotiaeva, N., Awasthi, S. K., and Good, L. (2005). Inhibition of Mycobacterium smegmatis gene expression and growth using antisense peptide nucleic acids. J. Mol. Microbiol. Biotechnol. 9, 101-109.
Kurupati, P., Tan, K. S., Kumarasinghe, G., and Poh, C. L. (2007). Inhibition of gene expression and growth by antisense peptide nucleic acids in a multiresistant beta-lactamase-producing Klebsiella pneumoniae strain. Antimicrob. Agents Chemother. 51, 805-811.
McGhee, G. C., Guasco, J., Bellomo, L. M., Blumer-Schuette, S. E., Shane, W. W., Irish-Brown, A., and Sundin, G. W. (2011). Genetic analysis of streptomycin-resistant (Sm(R)) strains of Erwinia amylovora suggests that dissemination of two genotypes is responsible for the current distribution of Sm(R) E. amylovora in Michigan. Phytopathology 101, 182-191.
Miller, T. D., and Schroth, M. N. (1972). Monitoring the epiphytic populations of Erwinia amylovora on pear with a selective medium. Phytopathology 62, 1175-1182.
Nakashima, N., Goh, S., Good, L., and Tamura, T. (2012). Multiple-gene silencing using antisense RNAs in Escherichia coli. Methods Mol. Biol. 815, 307-319.
Nekhotiaeva, N., Awasthi, S. K., Nielsen, P. E., and Good, L. (2004). Inhibition of Staphylococcus aureus gene expression and growth using antisense peptide nucleic acids. Mol. Ther. 10, 652-659.
Nikravesh, A., Dryselius, R., Faridani, O. R., Goh, S., Sadeghizadeh, M., Behmanesh, M., Ganyu, A., Klok, E. J., Zain, R., and Good, L. (2007). Antisense PNA accumulates in Escherichia coli and mediates a long post-antibiotic effect. Mol. Ther. 15, 1537-1542.
Norelli, J. L., Jones, A. L., and Aldwinckle, H. S. (2003). Fire blight management in the twenty-first century: using new technologies that enhance host resistance in apple. Plant Dis. 87, 756-765.
Rasmussen, L. C., Sperling-Petersen, H. U., and Mortensen, K. K. (2007). Hitting bacteria at the heart of the central dogma: sequence-specific inhibition. Microbial Cell Factories 6, 24.
Russo, N. L., Burr, T. J., Breth, D. I., and Aldwinckle, H. S. (2008). Isolation of streptomycin-resistant isolates of Erwinia amylovora in New York. Plant Dis. 92, 714-718.
Sundin, G. W., and Bender, C. L. (1996). Dissemination of the strA-strB streptomycin-resistance genes among commensal and pathogenic bacteria from humans, animals, and plants. Mol. Ecol. 5, 133-143.
Sundin, G. W., Castiblanco, L. F., Yuan, X., Zeng, Q., and Yang, C. H. (2016). Bacterial disease management: Challenges, experience, innovation, and future prospects. Mol. Plant Pathol. doi: 10.1111/mpp.12436
Sundin, G. W., Werner, N. A., Yoder, K. S., and Aldwinckle, H. S. (2009) Field evaluation of biological control of fire blight in the eastern United States. Plant Dis. 93, 386-394.
Tan, X. X., Actor, J. K., and Chen, Y. (2005). Peptide nucleic acid antisense oligomer as a therapeutic strategy against bacterial infection: proof of principle using mouse intraperitoneal infection. Antimicrob. Agents. Chemother. 49, 3203-3207.
Tsiantos, J., Psallidas, P., and Chatzaki, A. (2003) Efficacy of alternatives to antibiotic chemicals for the control of fire blight of pears. Ann. Appl. Biol. 143, 319-323.
van der Zwet, T., Orolaza-Halbrendt, N., and Zeller, W. (2012). Fire blight history, biology, and management. American Phytopathological Society Press.
Wang, G., and Xu, X. S. (2004). Peptide nucleic acid (PNA) binding-mediated gene regulation. Cell Res. 14, 111-116.
Wu, R. P., Youngblood, D. S., Hassinger, J. N., Lovejoy, C. E., Nelson, M. H., Iversen, P. L., and Moulton, H. M. (2007). Cell-penetrating peptides as transporters for morpholino oligomers: effects of amino acid composition on intracellular delivery and cytotoxicity. Nucleic Acids Res. 35, 5182-5191.
Yang, F., Korban, S. S., Pusey, P. L., Elofsson, M., Sundin, G. W., and Zhao, Y. (2014). Small-molecule inhibitors suppress the expression of both type III secretion and amylovoran biosynthesis genes in Erwinia amylovora. Mol. Plant Pathol. 15, 44-57.
Zatsepin, T. S., Turner, J. J., Oretskaya, T. S., and Gait, M. J. (2005). Conjugates of oligonucleotides and analogues with cell penetrating peptides as gene silencing agents. Curr. Pharm. Des. 11, 3639-3654.
Zeng, Q., McNally, R. R., and Sundin, G. W. (2013). Global small RNA chaperone Hfq and regulatory small RNAs are important virulence regulators in Erwinia amylovora. J. Bacteriol. 195, 1706-1717.
Zeng, Q., and Sundin, G. W. (2014). Genome-wide identification of Hfq-regulated small RNAs in the fire blight pathogen Erwinia amylovora discovered small RNAs with virulence regulatory function. BMC Genomics 15, 414.
Zhang, Y., and Cronan, J. E., Jr. (1998). Transcriptional analysis of essential genes of the Escherichia coli fatty acid biosynthesis gene cluster by functional replacement with the analogous Salmonella typhimurium gene cluster. J. Bacteriol. 180, 3295-3303.
Zhao, Y., Blumer, S. E., and Sundin, G. W. (2005) Identification of Erwinia amylovora genes induced during infection of immature pear tissue. J. Bacteriol. 187, 8088-8103.

### Example 2—Evaluation of the Effectiveness of Different Essential Genes Targeted by PNA-CPP

### Methods

10-bp oligomers of PNA-CPP1 with antisense sequences targeting the start codon region (−5 to +5) of six essential genes discovered in E. coli (Table 4) were tested for their effect in inhibiting the growth of E. amylovora. These compounds were serial diluted (20 μM, 4 μM, 0.8 μM, 0.16 μM), and were added into LB broth containing 5×10⁵ CFU/ml E. amylovora. The growth curve (OD600) was measured using a 96 well plate reader.

TABLE 4
PNA-CPPs Targeting Essential Genes in E. amylovora

| Gene | Function | Sequence | SEQ ID NO: |
|---|---|---|---|
| acpP | Acyl carrier protein | (KFF)3K-egl-ctcatactat-3′ | 2 and 1 |
| fabA | 3-hydroxydecanoyl-ACP | (KFF)3K-egl-accatattct-3′ | 2 and 19 |
| | dehydratase | | |
| fabD | malonyl coenzyme A acyl | (KFF)3K-egl-gtcattgtta-3′ | 2 and 20 |
| | carrier protein transacylase | | |
| ftsH | cell division protein | (KFF)3K-egl-gccatgtcac-3′ | 2 and 21 |
| gyrA | DNA gyrase subunit A | (KFF)3K-egl-ctcatggacg-3′ | 2 and 22 |
| glnA | glutamine synthetase | (KFF)3K-egl-gacatactta-3′ | 2 and 23 |

Bold indicate the sequence of PNA.

### Results

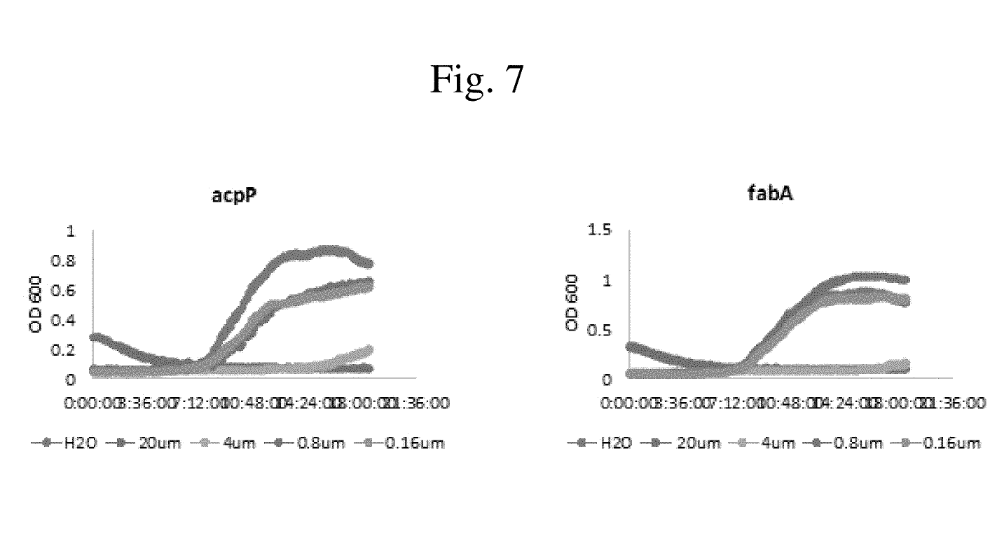

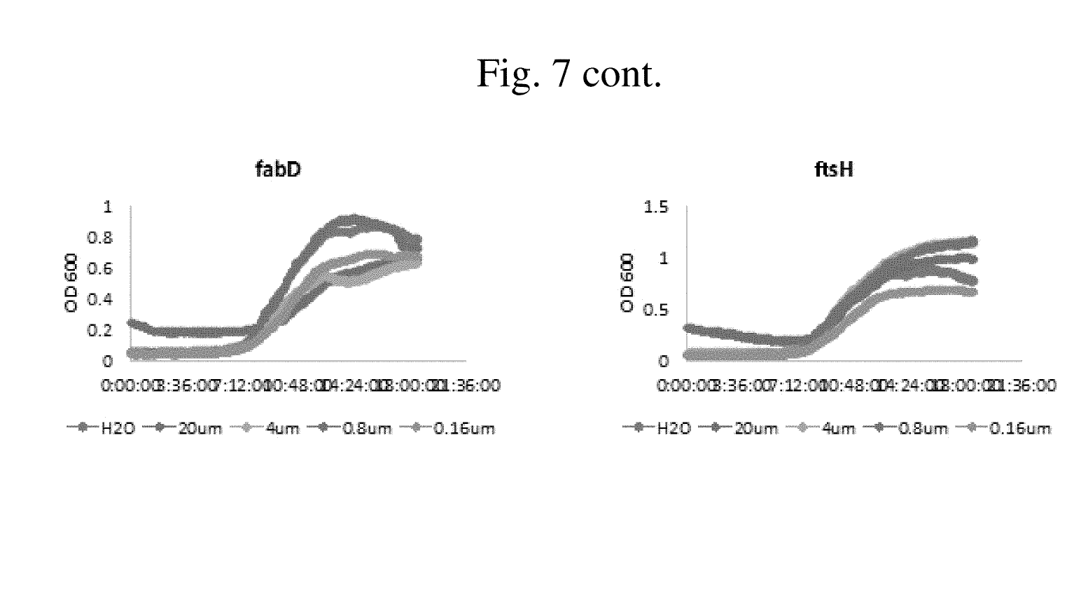

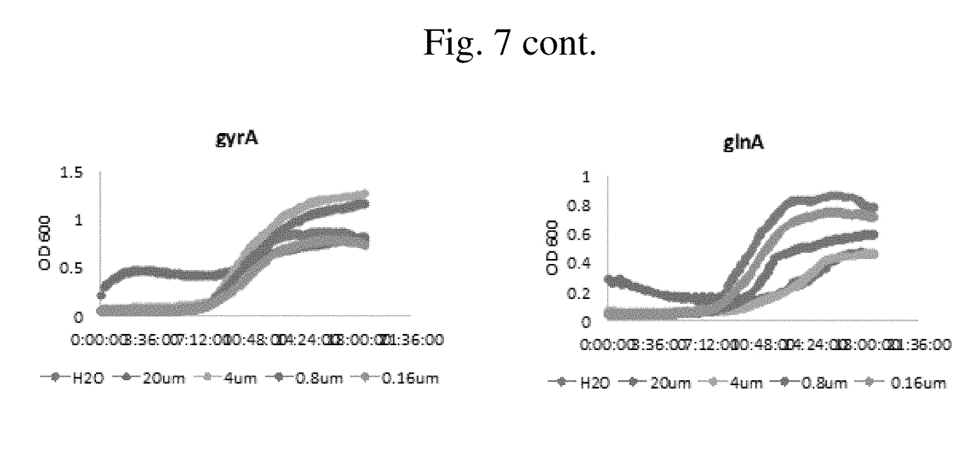

The addition of two antisense molecules, acpP (encoding an acyl carrier protein) and fabA (encoding a 3-hydroxydecanoyl-ACP dehydratase), caused growth inhibition of E. amylovora (FIG. 7). The minimal inhibition concentration is determined to be at 4 μM, for both acpP and fabA. The silencing of other genes did not seem to have any effect on the growth of E. amylovora at the concentrations tested (FIG. 7).

1. An antimicrobial composition comprising a cell penetrating peptide (CPP) linked to an antisense polynucleotide complementary to a target sequence in an RNA expressed from an essential gene in an Erwinia species.

2. The antimicrobial composition of claim 1, where in the essential gene is selected from the group consisting of acpP and fabA.

3. The antimicrobial composition of claim 1, wherein the cell penetrating peptide (CPP) is linked to the antisense polynucleotide via a linker.

4. The antimicrobial composition of claim 1, wherein the antisense polynucleotide is selected from the group consisting of a peptide nucleic acid (PNA), a phosphorodimidate mopholino oligomer (PMO), and a phosphorothioate oligonucleotide (PS-ODN).

5. The antimicrobial composition of claim 1, wherein the antisense polynucleotide comprises a peptide nucleic acid (PNA).

6. The antimicrobial composition of claim 1, wherein the target sequence comprises a start codon or a Shine-Dalgarno (SD) sequence.

7. The antimicrobial composition of claim 1, wherein the antisense polynucleotide consists of 6 to 20 nucleotides.

8. The antimicrobial composition of claim 1, wherein the antisense polynucleotide comprises SEQ ID NO: 1 (anti-acpP PNA sequence) or a fragment consisting of at least 6 nucleotides thereof.

9. The antimicrobial composition of claim 1, wherein the CPP is selected from the group consisting of SEQ ID NO: 2 (CPP1), SEQ ID NO: 3 (CPP2), and SEQ ID NO: 4 (CPP3).

10. The antimicrobial composition of claim 1, wherein the CPP comprises SEQ ID NO: 2 (CPP1).

11. The antimicrobial composition of claim 1, wherein the antimicrobial composition comprises SEQ ID NO: 1 and 2 (anti-acpP-CPP1), SEQ ID NO: 1 and 3 (anti-acpP-CPP2), SEQ ID NO: 1 and 4 (anti-acpP-CPP3), SEQ ID NO: 19 and SEQ ID NO: 2 (anti-fabA-CPP1), SEQ ID NO: 19 and SEQ ID NO:3 (anti-fabA-CPP2) or SEQ ID NO: 19 and SEQ ID NO: 4 (anti-fabA-CPP3).

12. The antimicrobial composition of claim 1, wherein the antimicrobial composition comprises SEQ ID NO: 1 linked to SEQ ID NO: 2 via an ethylene glycol linker (anti-acpP-CPP1) or SEQ ID NO: 19 linked to SEQ ID NO: 2 via an ethylene glycol linker.

13. An agricultural composition comprising the antimicrobial composition of claim 1 and a carrier, a biocontrol agent, or both.

14. The agricultural composition of claim 13, wherein the carrier comprises a nonionic surfactant.

15. The agricultural composition of claim 13, wherein the agricultural composition comprises a biocontrol agent selected from the group consisting of Bacillus subtilis strain QST 713, Bacillus amyloliquefaciens strain D747, Pantoea vagans C9-1, Pantoea, agglomerans strain E325, Pseudomonas fluorescens strain A506, and Aureobasidium pullulans strain DSM 14941 and 14942.

16. The agricultural composition of claim 13, wherein the concentration of the antimicrobial composition is 1 μM to 500 μM.

17. A method for inhibiting the growth of an Erwinia species on a plant comprising contacting the plant with an effective amount of the compositions of claim 1 to inhibit the growth of the Erwinia species on the plant.

18. The method of claim 17, wherein the plant is within the family Rosaceae.

19. The method of claim 17, wherein the plant is selected from the group consisting of an apple tree and a pear tree.

20. The composition one of claim 1, wherein the Erwinia species comprises Erwinia amylovora.


SEQUENCE LISTING (23 sequences)

SEQ ID NO: 1 - DNA, 10 bp, Artificial Sequence
  Synthetic anti-acpP peptide nucleic acid (PNA) sequence
ctcatactat

SEQ ID NO: 2 - protein, 10 aa, Artificial Sequence
  Synthetic cell penetrating peptide 1 (CPP1) sequence
KFFKFFKFFK

SEQ ID NO: 3 - protein, 14 aa, Artificial Sequence
  Synthetic cell penetrating peptide 2 (CPP2) sequence
  MOD_RES (1)..(1): Beta-alanine
  MISC_FEATURE (2)..(13): Any Xaa is 6-aminohexanoic acid
AXRXRRXRRXRRXR

SEQ ID NO: 4 - protein, 23 aa, Artificial Sequence
  Synthetic cell penetrating peptide 3 (CPP3) sequence
YARVRRRGPRGYARVRRRGPRRC

SEQ ID NO: 5 - DNA, 21 bp, Artificial Sequence
  Synthetic acpP forward primer
tgggcgttaagcaggaagaag

SEQ ID NO: 6 - DNA, 20 bp, Artificial Sequence
  Synthetic acpP reverse primer
tacgcctggtgaccattgat

SEQ ID NO: 7 - DNA, 20 bp, Artificial Sequence
  Synthetic amsk120F primer
catgcaatttccagtttcct

SEQ ID NO: 8 - DNA, 20 bp, Artificial Sequence
  Synthetic amsk120R primer
gcatgacggttaaccaaatc

SEQ ID NO: 9 - DNA, 22 bp, Artificial Sequence
  Synthetic amsk120Probe primer
tgcgtgacctgattcagcacaa

SEQ ID NO: 10 - DNA, 21 bp, Artificial Sequence
  Synthetic 63f primer
caggcctaacacatgcaagtc

SEQ ID NO: 11 - DNA, 18 bp, Artificial Sequence
  Synthetic 1387r primer
gggcggwgtgtacaaggc

SEQ ID NO: 12 - DNA, 10 bp, Artificial Sequence
  Synthetic Nontarget PNA
accatggtgg

SEQ ID NO: 13 - RNA, 23 nt, Erwinia amylovora
  misc_feature (1)..(23): Erwinia amylovora acpP start codon sequence
auuuaauaguaugagcacuaucg

SEQ ID NO: 14 - RNA, 23 nt, Pseudomonas putida
  misc_feature (1)..(23): Pseudomonas putida acpP start codon sequence
aaaacuagguaugagcaccaucg

SEQ ID NO: 15 - RNA, 23 nt, Pseudomonas fluorescens
  misc_feature (1)..(23): Pseudomonas fluorescens acpP start codon sequence
uaaacaagguaugagcaccaucg

SEQ ID NO: 16 - RNA, 23 nt, Pantoea agglomerans
  misc_feature (1)..(23): Pantoea agglomerans acpP start codon sequence
auuaaauaguaugagcgauaucg

SEQ ID NO: 17 - RNA, 23 nt, Xanthomonas euvesicatoria
  misc_feature (1)..(23): Xanthomonas euvesicatoria acpP start codon sequence
gauauccccaaugagcaccaucg

SEQ ID NO: 18 - RNA, 23 nt, Bacillus subtilis
  misc_feature (1)..(23): Bacillus subtilis acpP start codon sequence
gugaauugcuauggcagacacau

SEQ ID NO: 19 - DNA, 10 bp, Artificial Sequence
  Synthetic peptide nucleic acid (PNA) sequence
accatattct

SEQ ID NO: 20 - DNA, 10 bp, Artificial Sequence
  Synthetic peptide nucleic acid (PNA) sequence
gtcattgtta

SEQ ID NO: 21 - DNA, 10 bp, Artificial Sequence
  Synthetic peptide nucleic acid (PNA) sequence
gccatgtcac

SEQ ID NO: 22 - DNA, 10 bp, Artificial Sequence
  Synthetic peptide nucleic acid (PNA) sequence
ctcatggacg

SEQ ID NO: 23 - DNA, 10 bp, Artificial Sequence
  Synthetic peptide nucleic acid (PNA) sequence
gacatactta